(19) United States
(12) Patent Application Publication
(10) Pub. No.: US 2016/0367474 A1
(43) Pub. Date: Dec. 22, 2016

(54) TREATMENT PROCESS FOR SKIN IMPERFECTIONS

(71) Applicant: Anthony Pignataro, West Seneca, NY (US)

(72) Inventor: Anthony Pignataro, West Seneca, NY (US)

(21) Appl. No.: 14/952,120

(22) Filed: Nov. 25, 2015

Related U.S. Application Data
(60) Provisional application No. 62/180,764, filed on Jun. 17, 2015.

(51) Int. Cl. A61K 8/98; A61Q 19/08; B04B 5/04; B04B 9/02; A61B 17/32; A61F 13/40

(52) U.S. Cl. CPC A61K 8/983; A61B 17/32; A61M 35/006; B04B 5/0414; B04B 9/02; A61Q 19/08; A61B 2017/320004; A61B 2017/00761

(57) ABSTRACT

A method of treating a skin defect of a user is provided. The skin defect may include a wrinkle or a pimple on the user's face. After performing a dermabrasion of the affected area of skin, a blood sample is obtained from the user. The blood sample is then placed within a capsule containing a small amount of solvent. A supernatant of the blood sample is formed within the capsule via centrifugation. The supernatant is then applied to the skin defect of the user in an amount effective to treat the skin defect of the user.

### CROSS-REFERENCE TO RELATED APPLICATION

This application claims the benefit of priority of U.S. provisional application No. 62/180,764, filed Jun. 17, 2015, the contents of which are herein incorporated by reference.

### BACKGROUND OF THE INVENTION

The present invention relates to a skin care process and, more particularly, to a skin care process for the treatment of acne and wrinkles.

A pimple, zit or spot is a kind of comedo and one of the many results of excess oil getting trapped in the pores. Some of the varieties are pustules or papules. Pimples can be treated by various acne medications prescribed by a physician, or purchased at a pharmacy with a wide variety of treatments. A wrinkle, also known as a rhytid, is a fold, ridge or crease in the skin. Age wrinkling in the skin is promoted by habitual facial expressions, aging, sun damage, smoking, poor hydration, and various other factors. Current treatments of pimples and wrinkles include lotions that provide limited results.

As can be seen, there is a need for an improved treatment for improving pimples and wrinkles.

### SUMMARY OF THE INVENTION

In one aspect of the present invention, a method of treating a skin defect comprises: obtaining a blood sample from a user; placing the blood sample within a capsule containing a small amount of solvent; forming a supernatant of the blood sample within the capsule; and applying the supernatant to the skin defect of the user in an amount effective to treat the skin defect of the user.

These and other features, aspects and advantages of the present invention will become better understood with reference to the following drawings, description and claims.

### BRIEF DESCRIPTION OF THE DRAWINGS

FIG. 1 is a schematic view of an embodiment of the present invention; and

FIG. 2 is a flow chart of an embodiment of a process of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a skin care process. Facial acne and wrinkles are universal skincare issues that individuals struggle with. The skin care process of the present invention is used to treat these skin condition(s). The process may include a dermabrasion performed to the affected skin. A single drop of autologous blood is obtained by using a standard finger-stick device. Use the lancet to prick the tip of any finger to obtain a droplet of one's own blood. Add this droplet of blood to the 0.5 ml tubule containing approximately 0.35 ml-0.4 ml of solvent. The tubule is then capped and placed in the personal mini-centrifuge. The centrifuge is turned on and the capsule is spun for about 5 minutes. After the sediment has settled to the bottom, use a cotton tipped applicator to apply the supernatant to the affected skin. Whether used for acne or rhytids (wrinkles), it is the exfoliation/dermabrasion wound in which the plasma-based treatment leads to superficial wound antiseptics and the stimulation of tissue regeneration (wrinkle repair).

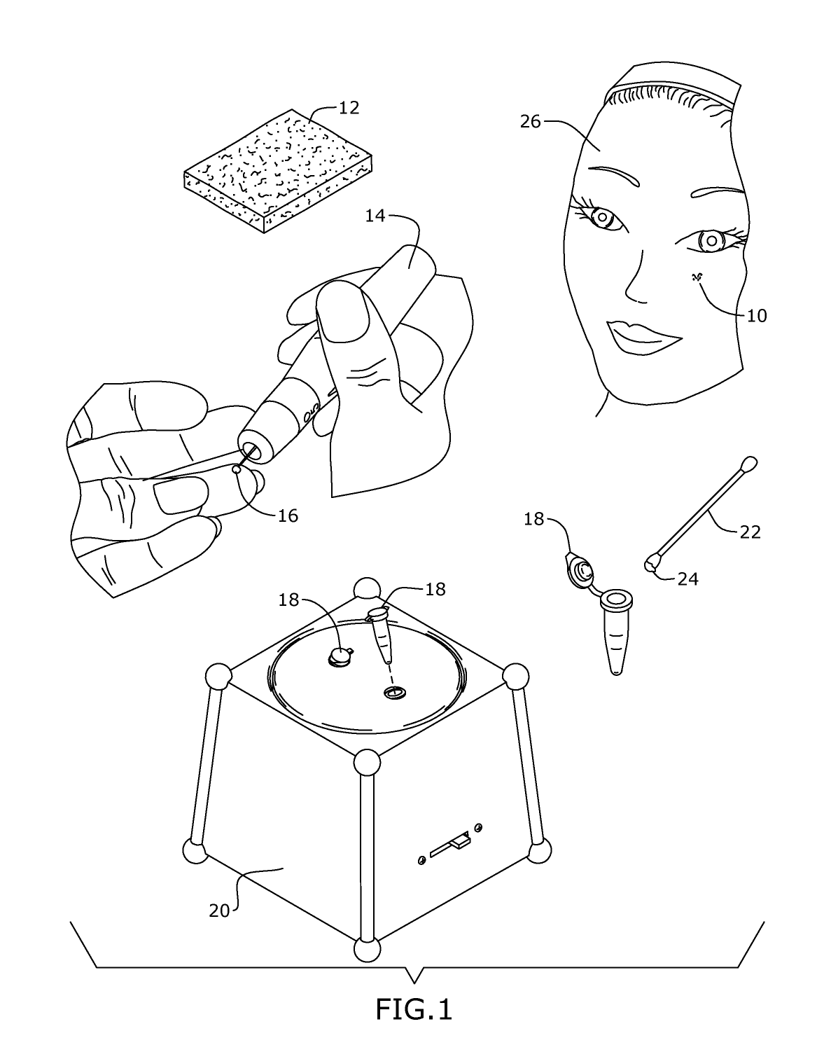

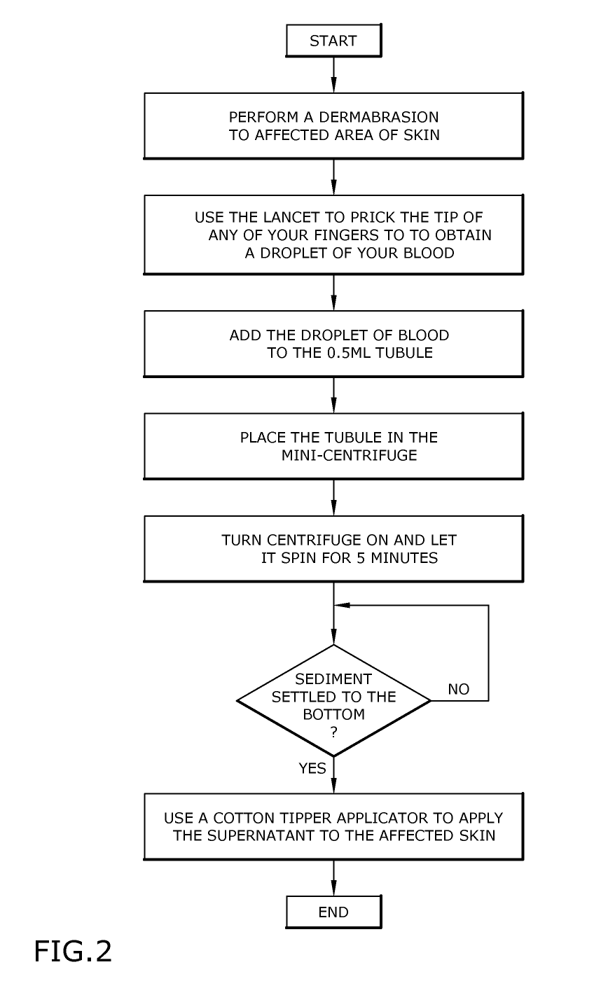

Referring to FIGS. 1 and 2, the present invention includes a method of treating a skin defect 10 of a user 26. The skin defect 10 may include a wrinkle or a pimple on the user's face. A blood sample 16 is obtained from the user 26. The blood sample 16 is then placed within a capsule 18 combined with a solvent. A supernatant 24 of the blood sample 16 is formed within the capsule. The supernatant 24 is then applied to the skin defect 10 of the user 26 in an amount effective to treat the skin defect 10 of the user 26.

The solvent of the present invention may include a saline solution. The saline solution may include about 0.9% sodium chloride in water. However, it is also contemplated that varying and different solutions/solvents may be used in this process as well. Many chemicals, minerals and/or elements can be used alone or bonded with other chemicals, minerals and/or elements to achieve a therapeutic effect when used in the process of the present invention. For example the solvent may include, but is not limited to, magnesium, zinc, sulfur, calcium and the like. Potential elements may be put into a solution either alone or in combination with other elements. For example, magnesium chloride, magnesium sulfate, calcium chloride and many other potential elemental combinations are potentially useful and therapeutic when used as a solvent in this process. Further, the concentration of the solvent/solution may be altered and a thickener may be used to change (thicken) the consistency of a given solution in any concentration. Examples of such thickeners may include, but are not limited to, Glucamate CCO, Xanthan gum, and the like.

In certain embodiments, the supernatant 24 of the blood sample is formed using a centrifuge 20. The centrifuge 20 may include a miniature centrifuge 20 having no more than two openings, each sized to receive the capsule 18 within. The capsule 18 with the blood sample 16 is placed within the centrifuge 20 and the centrifuge 20 is turned on. The capsule 18 is spun for about 5 minutes or until sediment has settled to the bottom of the capsule 18. The supernatant 24 formed above the sediment is then applied to the skin.

The centrifuge 20 of the present invention may include a plastic six-sided pyramidal box or casing, which has a flat top and bottom. The bottom or base of the centrifuge 20 may be wider than the top. The centrifuge may include a battery terminal for two AA batteries mounted in the base of the casing. A hinged door may provide access to the battery terminal for replacing batteries. The centrifuge 20 may further include a small internal electric motor and an internal mounting bracket for the motor. A circular spinning disc has two small ports sized to accept the 0.5 ml tubules. Copper wiring is insulated and is of sufficient length to connect the motor to an on/off switch and the battery terminal.

The present invention may further include the step of performing a dermabrasion to the skin defect 10 prior to applying the supernatant 24. In such embodiments, the present invention may include a dermabrasion pad 12. The dermabrasion pad 12 is rubbed against the skin defect 10, and then the supernatant 24 is applied to the defect 10.

Obtaining the blood sample 16 from the user 26 may include pricking a finger tip with a lancet 14 to form a puncture of the skin. A blood droplet is then extracted from the punctured skin and is used as the blood sample 16.

The step of applying the supernatant to the skin defect may include swabbing the supernatant with a cotton swab. In certain embodiments, the cotton swab may include the cotton swab of a cotton tipped applicator 22. The cotton swab of the cotton tipped applicator 22 is then rubbed against the skin defect 10, thereby applying the supernatant to the skin defect 10.

As designed, the present invention invokes the healing and regenerative properties of one's own plasma. The present invention may be applied to the skin defect twice a week or may be dependent on the condition and the tolerance of the user. Further, there could be additional uses and indications for the use of one's autologous plasma. Further, it is possible that other and different solvents could be added to the tubule prior to centrifugation resulting in other products which may have different indications/uses.

It should be understood, of course, that the foregoing relates to exemplary embodiments of the invention and that modifications may be made without departing from the spirit and scope of the invention as set forth in the following claims.

What is claimed is:

1. A method of treating a skin defect comprising:
obtaining a blood sample from a user;
placing the blood sample within a capsule;
forming a supernatent of the blood sample within the capsule; and
applying the supernatant to the skin defect of the user in an amount effective to treat the skin defect of the user.

2. The method of claim 1, wherein the step of forming the supernatant comprises the steps of:
placing the capsule within a centrifuge;
operating the centrifuge until a sediment has settled at the bottom of the capsule.

3. The method of claim 2, wherein the centrifuge comprises a miniature centrifuge comprising no more than two openings, each of the openings sized to receive the capsule.

4. The method of claim 1, further comprising the step of:
performing a dermabrasion to the skin defect prior to applying the supernatant.

5. The method of claim 4, wherein a dermabrasion pad is used to perform the dermabrasion.

6. The method of claim 1, wherein the step of obtaining a blood sample from a user comprises the steps of:
pricking a finger tip of the user with a lancet forming a puncture; and
extracting a blood droplet from the puncture.

7. The method of claim 1, wherein the step of applying the supernatant to the skin defect comprises the steps of:
swabbing the supernatant with a cotton swab from the capsule; and
rubbing the cotton swab against the skin defect.

8. The method of claim 1, wherein the skin defect comprises at least one of a pimple and a wrinkle.

9. The method of claim 1, further comprising the step of mixing a solvent with the blood sample, wherein the mixture is within the capsule.

10. The method of claim 9, wherein the solvent is a saline solution.
